(19) United States
(12) Patent Application Publication
(10) Pub. No.: US 2009/0049722 A1
(43) Pub. Date: Feb. 26, 2009

(54) WRISTBAND WITH BLOOD-TYPE INFORMATION

(75) Inventor: Yuk Yee CHAN, Hong Kong (HK)

Correspondence Address: EGBERT LAW OFFICES, 412 MAIN STREET, 7TH FLOOR, HOUSTON, TX, 77002, US

(73) Assignee: AGOG LIMITED, Hong Kong (CN)

(21) Appl. No.: 11/841,662

(22) Filed: Aug. 20, 2007

(51) Int. Cl. A61B 5/117

(52) U.S. Cl. 40/633; 63/3

(57) ABSTRACT

The present invention is a wristband with blood-type information in Braille, words, symbols, graphics or anything alike capable of bearing such information raised on, recessed, or printed on the surface of the wristband. The wristband is made of elastic materials or a combination of other materials to form a flexible closed-end loop. The wristband is manufactured in different sizes or is adjustable to fit different wrist sizes. The wristband is water-proof or splash-proof. The wristbands are manufactured in different colors or combinations of colors. The wristband allows everyone, whether they have normal sight or vision problems, whether they are a child or elderly, and whether they are male or female, without any restriction.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to instrument for blind individuals and normally-sighted persons, in particular, a wristband with blood-type information.

2. Description of Related Art Including Information Disclosed Under 37 CFR 1.97 and 37 CFR 1.98

There are various prior art devices designed for blind individuals to take care of their daily needs. The known prior art discloses an application of a wristband for medical use, wherein the medical information of the wearer, including blood-type information, is provided for both blind and normally-sighted people. However, the information is too technical and only for use in hospitals. Blind individuals still need something that is easy to use for carrying their blood-type information in case there is an emergency. The blood-type information so carried, on the other hand, must also be easily readable by normally-sighted persons. The blood-type information in the form of a wristband can be a sort of fashion accessory, an ornamental piece, etc.

### BRIEF SUMMARY OF THE INVENTION

The present invention is a wristband carrying blood-type information. The blood-type information is in the form of Braille, symbols, words, graphics, colors and/or anything alike raised above, recessed, printed on the outer surface of the wristband. The purpose of the Braille is to allow the blind to read their blood-type information by touch. The same blood-type information is also presented in words, symbols or graphics to allow the non-blind individuals to easily read the information. The wristband is a flexible close-end loop. It is made of elastic materials or a combination of other materials with elastic materials which can be easily extended and worn on wrist. It also serves as a fashion accessories and/or personal ornamentation, similar to a wristband or watch. The wearer could take it daily as part of daily life activity. It is made of elastic material, and it is water-proof in order to provide maximum comfort and durability. The wristband is manufactured in different sizes or is adjustable to fit different wrist sizes, fitting children and the elderly, men and women. The wristband is water-proof or splash-proof so that it can be worn during outdoor or water activities. The wristbands are manufactured in different colors or combination of colors to make them part of fashion. The wristband carries the blood type information in the form of visually identifiable words, symbols, graphics or colors, as well as in Braille. Therefore, the wristband allows everyone access, whether having normal sight or vision problems, whether a child or elderly, and whether male or female, without any restriction.

### BRIEF DESCRIPTION OF THE SEVERAL VIEWS OF THE DRAWINGS

The accompanying drawings, which are incorporated in and constitute a part of this disclosure, illustrate various embodiments and aspects of the present invention.

FIG. 1 shows two perspective view of one embodiment of the present invention with Blood-type AB+ in Braille on one side and in words on an opposite side.

FIG. 2 shows schematic views of various embodiments of the present invention with different blood-type information.

### DETAILED DESCRIPTION OF THE INVENTION

The following detailed description refers to the accompanying drawings. Wherever possible, the same reference numbers are used in the drawings and the following description to refer to the same or similar parts. While several exemplary embodiments and features of the present invention are described herein, modifications, adaptations and other implementations are possible, without departing from the spirit and scope of the invention. For example, substitutions, additions or modifications may be made to the components illustrated in the drawings, and the exemplary methods described herein may be modified by substituting, reordering or adding steps to the disclosed methods. Accordingly, the following detailed description does not limit the present invention. Instead, the proper scope of the present invention is defined by the appended claims.

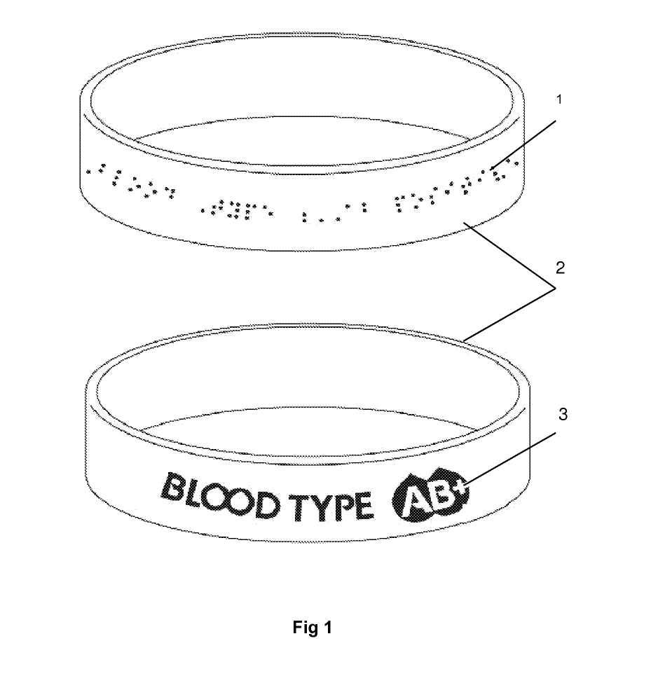

The present invention is a wristband carrying blood-type information. In one embodiment as shown in FIG. 1, the blood-type 1 is presented in form of Braille raised above one side of the outer surface of the wristband 2. The same blood-type information is also presented in raised, recessed or printed words 3 above the other side of the outer surface of the wristband 2. The Braille allows a blind person to read their blood-type information by touch with 3-D feel, while the words allow the non-blind individuals to easily read the information. The wristband 2 is a flexible close-end loop. It is made of elastic materials which can be easily extended and worn on a wrist.

Alternatively, the blood type information, in the form of Braille and words, and alike, can be raised on, recessed, or printed on the same side of the wristband, and even both sides of the wristband.

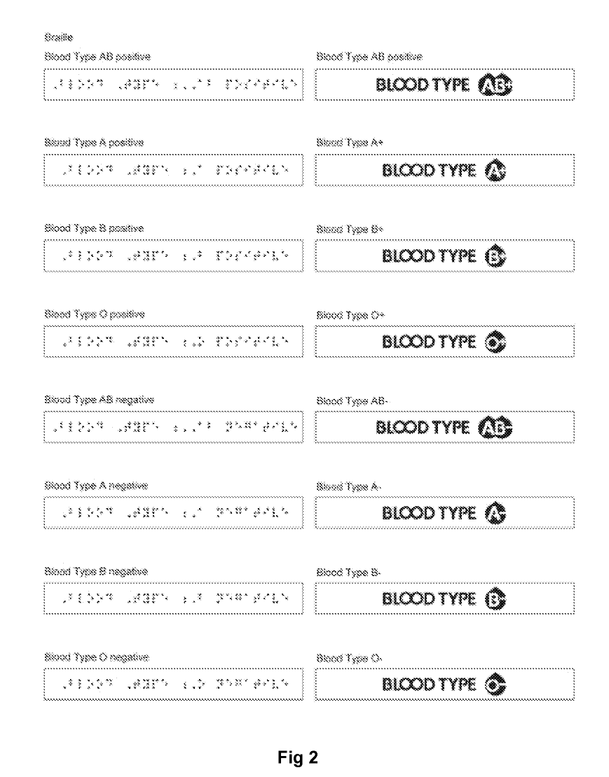

FIG. 2 shows other embodiments wherein different blood-types are presented. The same blood-type information is presented in both raised Braille and words on each side of the outer surface of a single wristband. Users can wear the wristband with their own blood-type.

The blood-type information is manufactured as raised Braille, words, symbols, graphics or anything together with the wristband or part of the wristband. In one embodiment, the wristband is made of elastic materials to form a flexible close-end loop. Users can easily extend the wristband and wear the wristband on their wrists. In another embodiment, the wristband is made of a combination of other materials with elastic materials. In preferred embodiments, the material elasticity allows the wearer to turn over the side of the wristband at will and easily.

The wristband is manufactured in different sizes or is adjustable to fit different wrist sizes. In one embodiment, the wristband is manufactured in small, medium and large sizes. In another embodiment, the wristband has adjustable parts to fit all wrist sizes.

The wristband has no removable parts, no slot and no detachable cinch or label.

The wristband is water-proof or splash-proof so that it can be wear during outdoor or water activities.

The wristbands are manufactured in different colors or combination of colors to make them part of the fashion.

While certain features and embodiments of the present invention have been described, other embodiments of the present invention will be apparent to those skilled in the art from consideration of the specification and practice of the embodiments of the invention disclosed herein. It is intended, therefore, that the specification and examples be considered as exemplary only, with a true scope and spirit of the present invention being indicated by the following claims and their full scope of equivalents.

1. A wristband with digital memory for electronics data storage primary with blood-type information;

2. The wristband in claim 1, wherein the digital memory for electronics data storage is powered by an internal battery or external power sources, stored data in the memory is comprised of a voice memo, multi-media files, personal information, or blood-type information.

3. The wristband in claim 1, wherein said electronics data as Braille, symbols, words, graphics or anything raised above, recessed, or printed on at least one side of a surface is legible by touch or by vision.

4. The wristband in claim 1, wherein the electronics data is transferred by data cable, wireless transfer, radio frequency, optic fiber, the electronics data being input from a data source, being input from and output to any kind of electronics data devices, computer, PDA, cell phones, etc. via various communication method.

5. The wristband in claim 1, wherein at least one part of digital memory electronics components is embedded.

6. The wristband in claim 1, being comprised of elastic materials or combination of other materials to form a flexible close-end loop.

7. The wristband in claim 1, wherein the wristband is manufactured in different sizes or is adjustable to fit different sizes.

8. The wristband in claim 1, wherein the wristband is water-proof or splash-proof.

9. The wristband in claim 1, wherein the wristband can be of one single color or more than one color.
